(19) United States
(12) Patent Application Publication
(10) Pub. No.: US 2021/0299339 A1
(43) Pub. Date: Sep. 30, 2021

(54) BLOOD MONITORING SYSTEM FOR DETERMINING A CALIBRATED HEMOGLOBIN CONCENTRATION VALUE FOR A PATIENT BASED ON PATIENT-SPECIFIC MEAN CORPUSCULAR HEMOGLOBIN CONCENTRATION DATA

(71) Applicant: Fresenius Medical Care Holdings, Inc., Waltham, MA (US)

(72) Inventor: Stephan Thijssen, New York, NY (US)

(21) Appl. No.: 16/836,721

(22) Filed: Mar. 31, 2020

(51) Int. Cl. A61M 1/16; G16H 10/60; G16H 10/40

(52) U.S. Cl. CPC A61M 1/1603; G16H 10/60; G16H 10/40; A61M 2230/20; A61M 2205/3584; A61M 2205/52; A61M 2205/3306; A61M 2230/207

(57) ABSTRACT

A hemodialysis system includes a hemodialysis machine and a blood monitoring system. The hemodialysis machine is configured to provide hemodialysis treatment to a patient, wherein the hemodialysis treatment includes circulating extracorporeal blood of the patient through an extracorporeal blood circuit. The blood monitoring system includes: a sensor device configured to measure a hematocrit value corresponding to the extracorporeal blood of the patient in the extracorporeal blood circuit; and at least one controller. The blood monitoring system is configured to communicate with an electronic health records (EHR) system over a communications network to obtain patient-specific mean corpuscular hemoglobin concentration (MCHC) data for the patient. The at least one controller is configured to determine a hemoglobin concentration value corresponding to the extracorporeal blood for the patient in the extracorporeal blood circuit using the measured hematocrit value and the patient-specific MCHC data for the patient.

### BACKGROUND

Patients with kidney failure or partial kidney failure typically undergo dialysis treatment in order to remove toxins and excess fluids from their blood. Hemodialysis is one of the common forms of dialysis treatment. To perform hemodialysis, blood is taken from a patient through an intake needle or catheter which draws blood from an arteriovenous vascular access or vein, respectively, located in a specifically accepted access location—for example, a shunt surgically placed in an arm, or a central-venous catheter placed in the superior vena cava. The needle or catheter is connected to extracorporeal tubing that is fed to a peristaltic pump and then to a dialyzer that cleans the blood and removes excess fluid. The cleaned blood is then returned to the patient through additional extracorporeal tubing typically connected to another needle in the arteriovenous vascular access or to a separate lumen of the central-venous catheter. Sometimes, a heparin drip is located in the hemodialysis loop to prevent the blood from coagulating.

As the drawn blood passes through the dialyzer, it travels in straw-like tubes within the dialyzer that serve as semi-permeable passageways for the unclean blood. Fresh dialysate solution enters the dialyzer at its downstream end. The dialysate surrounds the straw-like tubes and flows through the dialyzer in the opposite direction of the blood flowing through the tubes. Fresh dialysate collects toxins passing through the straw-like tubes by diffusion and excess fluids in the blood by ultra filtration. Dialysate containing the removed toxins and excess fluids is disposed of as waste. The red cells remain in the straw-like tubes and do not pass into the dialysate.

A blood monitoring system is often used during hemodialysis treatment or other treatments involving extracorporeal blood flow. The blood monitoring system may use optical techniques to non-invasively measure in real-time the hematocrit and the oxygen saturation level of blood flowing through the hemodialysis system. The blood monitoring system may measure the blood at, for example, a sterile blood chamber attached in-line to the extracorporeal tubing.

In general, blood chambers along with the tube set and dialyzer are replaced for each patient. The blood chamber is intended for a single use. The blood chamber defines an internal blood flow cavity comprising a substantially flat viewing region and two opposing viewing lenses. Emitters (such as light-emitting diode (LED) emitters) and photodetectors for the optical blood monitoring system are fastened (e.g., by clipping) into place onto the blood chamber over the lenses. Multiple wavelengths of light may be resolved through the blood chamber and the patient's blood flowing through the chamber with a photodetector detecting the resulting intensity of each wavelength.

The preferred wavelengths to measure hematocrit are about 810 nm, which is substantially isobestic for red blood cells, and about 1300 nm, which is substantially isobestic for water. A ratiometric technique may be used to calculate the patient's hematocrit value in real-time based on this light intensity information. The hematocrit value is a percentage determined by the ratio between (1) the volume of the red blood cells in a given whole blood sample and (2) the overall volume of the blood sample.

In a clinical setting, the actual percentage change in blood volume occurring during hemodialysis can be determined, in real-time, from the change in the measured hematocrit. Thus, an optical blood monitoring system is able to non-invasively monitor not only the patient's hematocrit level but also the change in the patient's blood volume in real-time during a hemodialysis treatment session. The ability to monitor real-time change in blood volume helps facilitate safe, effective hemodialysis.

To monitor blood in real-time, emitters and photodetectors may be mounted on two opposing heads of a sensor clip assembly that fits over a blood chamber. For accuracy of the system, the emitters and the photodetectors may be located in a predetermined position and orientation each time the sensor clip assembly is clipped into place over the blood chamber. The predetermined position and orientation ensure that light traveling from the emitters to the photodetectors travels through the lenses of the blood chamber.

The optical blood monitoring system may be calibrated for the specific dimensions of the blood chamber and the specific position and orientation of the sensor clip assembly relative to the blood chamber. For this purpose, the sensor clip assembly may be configured to mate to the blood chamber so that the emitters and the photodetectors are at a predetermined position and orientation relative to one another and to the blood chamber.

An example of an optical blood monitoring system having a sensor clip assembly configured to measure hematocrit and oxygen saturation of extracorporeal blood flowing through a blood chamber is described in U.S. Pat. No. 9,801,993, titled “SENSOR CLIP ASSEMBLY FOR AN OPTICAL MONITORING SYSTEM,” which is incorporated by reference in its entirety herein.

### SUMMARY

In an exemplary embodiment, the present application provides a hemodialysis system, comprising: a hemodialysis machine configured to provide hemodialysis treatment to a patient, wherein the hemodialysis treatment includes circulating extracorporeal blood of the patient through an extracorporeal blood circuit; and a blood monitoring system, comprising: a sensor device configured to measure a hematocrit value corresponding to the extracorporeal blood of the patient in the extracorporeal blood circuit; and at least one controller; wherein the blood monitoring system is configured to communicate with an electronic health records (EHR) system over a communications network to obtain patient-specific mean corpuscular hemoglobin concentration (MCHC) data for the patient; and wherein the at least one controller is configured to determine a hemoglobin concentration value corresponding to the extracorporeal blood for the patient in the extracorporeal blood circuit using the measured hematocrit value and the patient-specific MCHC data for the patient.

In a further exemplary embodiment, the at least one controller of the blood monitoring system comprises a controller disposed within the sensor device.

In a further exemplary embodiment, the blood monitoring system further comprises a display device configured for displaying the measured hematocrit value and the determined hemoglobin concentration value.

In a further exemplary embodiment, the at least one controller of the blood monitoring system comprises a controller disposed within the sensor device.

In a further exemplary embodiment, the patient-specific MCHC data for the patient comprises multiple MCHC values; the at least one controller is further configured to determine an average MCHC value from the multiple MCHC values; and determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

In a further exemplary embodiment, the patient-specific MCHC data for the patient comprises a most recent MCHC value determined for the patient based on laboratory analysis of a blood sample of the patient.

In a further exemplary embodiment, the patient-specific MCHC data for the patient comprises an average MCHC value determined for the patient based on laboratory analyses of multiple blood samples of the patient.

In a further exemplary embodiment, determining the hemoglobin concentration value corresponding to the extracorporeal blood of the patient in the extracorporeal blood circuit comprises performing a multiplication or division operation using the measured hematocrit value and an MCHC value corresponding to the patient-specific MCHC data for the patient.

In a further exemplary embodiment, the blood monitoring system is further configured to communicate with the EHR system to provide the determined hemoglobin concentration value to the EHR system.

In a further exemplary embodiment, the blood monitoring system further comprises a user interface for receiving input of a patient-specific MCHC value for the patient; and the at least one controller is further configured to determine a hemoglobin concentration value corresponding to the extracorporeal blood of the patient in the extracorporeal blood circuit using the measured hematocrit value and the input patient-specific MCHC value for the patient.

In a further exemplary embodiment, the blood monitoring system is configured to update the measured hematocrit value in real-time; and the at least one controller is configured to correspondingly update the determined hemoglobin concentration value in real-time.

In a further exemplary embodiment, the blood monitoring system is an optical blood monitoring system.

In another exemplary embodiment, the present application provides a method for monitoring blood using a blood monitoring system, comprising: measuring, by a sensor device of the blood monitoring system, a hematocrit value of extracorporeal blood of a patient in an extracorporeal circuit to which the sensor device is attached; obtaining, by at least one controller of the blood monitoring system, patient-specific mean corpuscular hemoglobin concentration (MCHC) data for the patient; determining, by the at least one controller of the blood monitoring system, a hemoglobin concentration value corresponding to the extracorporeal blood of the patient in the extracorporeal blood circuit using the measured hematocrit value and the patient-specific MCHC data for the patient; and outputting, by the at least one controller of the blood monitoring system, the determined hemoglobin concentration value.

In a further exemplary embodiment, obtaining the patient-specific MCHC data for the patient further comprises: obtaining, by at least one controller of the blood monitoring system, the patient-specific MCHC data from an electronic health records (EHR) system via a communications interface of the blood monitoring system.

In a further exemplary embodiment, the patient-specific MCHC data comprises multiple MCHC values; the method further comprises: determining, by the at least one controller of the blood monitoring system, an average MCHC value from the multiple MCHC values; and determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

In a further exemplary embodiment, the patient-specific MCHC data for the patient comprises a most recent MCHC value determined for the patient based on laboratory analysis of a blood sample of the patient.

In a further exemplary embodiment, the patient-specific MCHC data for the patient comprises an average MCHC value determined for the patient based on laboratory analyses of multiple blood samples of the patient.

In a further exemplary embodiment, the blood monitoring system is an optical blood monitoring system.

In yet another exemplary embodiment, the present application provides a non-transitory computer-readable medium having processor-executable instructions stored thereon for monitoring blood using a blood monitoring system, the processor-executable instructions, when executed, facilitating: measuring a hematocrit value of extracorporeal blood of a patient in an extracorporeal circuit to which the sensor device is attached; obtaining patient-specific mean corpuscular hemoglobin concentration (MCHC) data for the patient; determining a hemoglobin concentration value corresponding to the extracorporeal blood of the patient in the extracorporeal blood circuit using the measured hematocrit value and the patient-specific MCHC data for the patient; and outputting the determined hemoglobin concentration value.

In a further exemplary embodiment, obtaining the patient-specific MCHC data for the patient further comprises: obtaining the patient-specific MCHC data from an electronic health records (EHR) system via a communications interface of the blood monitoring system.

In a further exemplary embodiment, the patient-specific MCHC data comprises multiple MCHC values; the processor-executable instructions, when executed, further facilitate: determining an average MCHC value from the multiple MCHC values; and determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

In a further exemplary embodiment, the patient-specific MCHC data for the patient comprises a most recent MCHC value determined for the patient based on laboratory analysis of a blood sample of the patient.

In a further exemplary embodiment, the patient-specific MCHC data for the patient comprises an average MCHC value determined for the patient based on laboratory analyses of multiple blood samples of the patient.

In a further exemplary embodiment, the blood monitoring system is an optical blood monitoring system.

### BRIEF DESCRIPTION OF THE DRAWINGS

FIGS. 1A-1C are plots showing changes in average mean corpuscular volume (MCV), mean corpuscular hemoglobin (MCH) and mean corpuscular hemoglobin concentration (MCHC) values over time determined for a population of dialysis patients across multiple dialysis clinics.

FIG. 2 is a schematic diagram of an exemplary hemodialysis system having an optical blood monitoring system.

FIG. 3 is a block diagram of an exemplary network environment in which a hemodialysis system communicates with an electronic health records (EHR) system to obtain patient-specific MCHC data and determine a calibrated hemoglobin (Hgb) concentration value based on the patient-specific MCHC data.

FIG. 4A is a flowchart of an exemplary process for determining a calibrated Hgb concentration value for a patient using an optical blood monitoring system.

FIG. 4B is a flowchart of another exemplary process for determining a calibrated Hgb concentration value for a patient using an optical blood monitoring system.

FIG. 4C is a flowchart of another exemplary process for determining a calibrated Hgb concentration value for a patient using an optical blood monitoring system and an EHR system.

### DETAILED DESCRIPTION

Existing blood monitoring systems, such as described in U.S. Pat. No. 9,801,993, are able to provide accurate, real-time measurements of hematocrit (Hct). These Hct measurements may further be used to determine and output hemoglobin (Hgb) concentration values. The Hgb for a blood sample corresponds to the mass of protein (e.g., in grams) for the blood sample, and an Hgb concentration value corresponds to a protein mass per unit of blood sample volume. The Hgb concentration value may be determined based on multiplying an Hct value and a mean corpuscular hemoglobin concentration (MCHC) value. It will be appreciated that the Hct value corresponds to the volume of red blood cells (RBCs) in a blood sample divided by the total volume of the blood sample, and that the MCHC value corresponds to an average mass of Hgb per RBC divided by an average volume per RBC. It will further be appreciated that the MCHC value corresponds to mean corpuscular hemoglobin (MCH) divided by mean corpuscular volume (MCV), wherein MCH corresponds to an average mass of Hgb per RBC of a patient (e.g., in picograms), and wherein MCV corresponds to an average volume per RBC of a patient (e.g., in femtoliters). Thus, when the Hct value is multiplied by the MCHC value, the Hgb concentration value that is determined corresponds to a protein mass per unit of blood sample volume.

A shortcoming of existing blood monitoring systems, however, is that they use a default MCHC value for all patients when determining an Hgb concentration value for a respective patient. Thus, if a particular patient's actual MCHC is different from the default MCHC value used in the determination of the Hgb concentration value, the determined Hgb concentration value for that patient might not accurately reflect the actual Hgb concentration value for that patient.

For example, the default MCHC used in an existing blood monitoring system may be 34.0136 g/dl, and the blood monitoring system determines that an Hct for a blood sample of a patient is 0.33 (or 33%). In this situation, the blood monitoring system determines the Hgb concentration value for the patient as being 0.33*34.0136 g/dl (which may also be expressed as (33/100)*34.0136 g/dl, or as 0.33/(1/34.0136 g/dl)), resulting in a determined Hgb concentration value of 11.22 g/dl. However, if the patient's actual MCHC value is 31.37 g/dl, the actual Hgb concentration value for the patient should be 10.35 g/dl, which means that the blood monitoring system's determined Hgb concentration value is off by 0.87 g/dl. This is not a trivial deviation. A more accurate and reliable monitoring method will be advantageous and allow for improved treatment (e.g., of anemia management) in clinical practice.

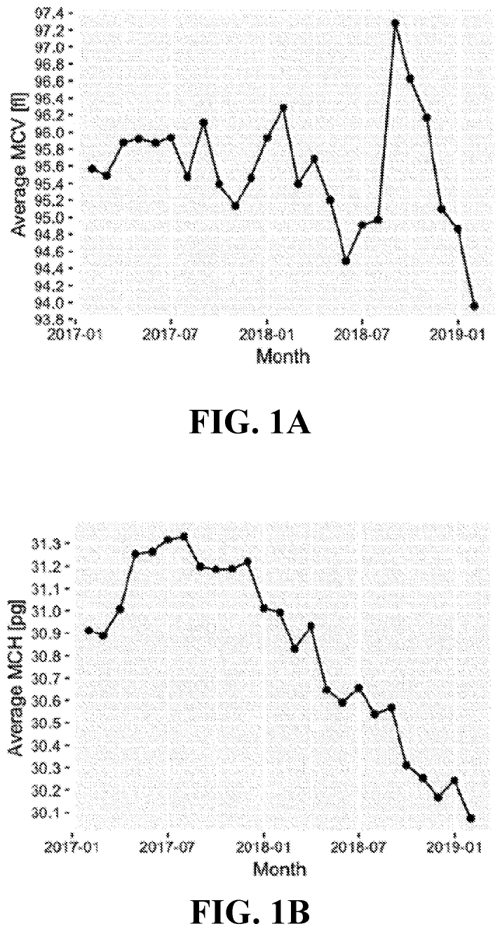

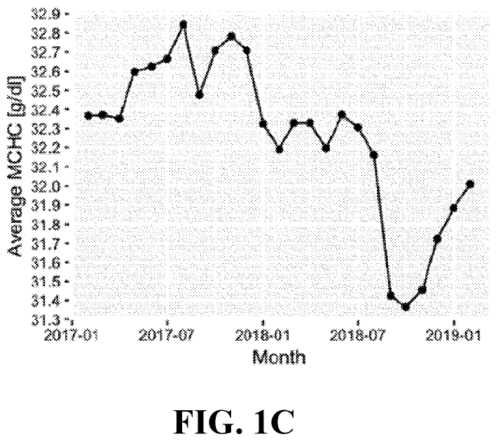

There are many factors that can affect MCHC, and a particular patient's MCHC value is not static. Nor is the average MCHC value across all patients static. For example, changes in anemia management practice patterns (such as changing a ratio between use of erythropoiesis stimulating agents (ESAs) and iron use) may affect the MCHCs of many patients, such that the average MCV, MCH and MCHC values across all patients are subject to various changes over time (in addition to individual MCV, MCH and MCHC values for a particular patient changing over time). Based on empirical data gathered from multiple dialysis clinics, it has been confirmed that MCV, MCH and MCHC for the patient population served by those clinics has significantly changed over time. For example, FIGS. 1A-1C are plots showing changes in average mean corpuscular volume (MCV), mean corpuscular hemoglobin (MCH) and mean corpuscular hemoglobin concentration (MCHC) values over time determined for a population of dialysis patients across multiple dialysis clinics.

Exemplary embodiments of the present application utilize individual, patient-specific MCHC values for respective patients to determine and output calibrated Hgb concentration values for the respective patients, such that reliability and accuracy of Hgb concentration values determined by blood monitoring systems is improved. For example, in an exemplary embodiment involving a hemodialysis treatment, a blood monitoring system of a hemodialysis system may obtain a patient-specific MCHC value for a particular patient from an EHR system or via user input. The blood monitoring system then uses the patient-specific MCHC value (together with a real-time Hct measurement for the patient) to determine a calibrated Hgb concentration value for the patient. In other words, by utilizing an individual, patient-specific MCHC value, a blood monitoring system in accordance with an exemplary embodiment of the present application is able to determine an Hgb concentration value for a patient in a manner that has been calibrated specifically for the patient.

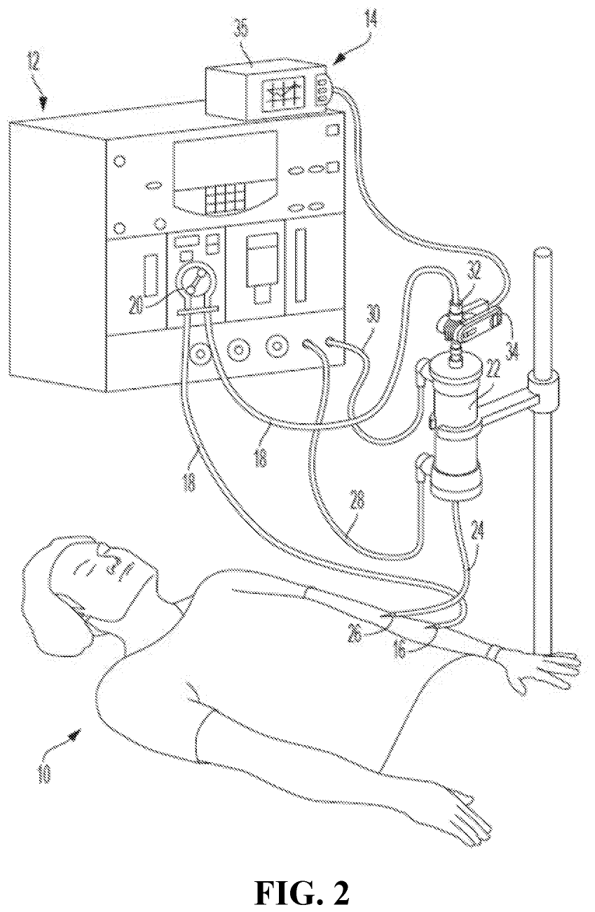

FIG. 2 is a schematic diagram of an exemplary hemodialysis system having an optical blood monitoring system. FIG. 2 depicts a patient 10 undergoing hemodialysis treatment using a hemodialysis machine 12. The hemodialysis system further includes an optical blood monitoring system 14.

An inlet needle or catheter 16 is inserted into an access site of the patient 10, such as in the arm, and is connected to extracorporeal tubing 18 that leads to a peristaltic pump 20 and to a dialyzer 22 (or blood filter). The dialyzer 22 removes toxins and excess fluid from the patient's blood. The dialyzed blood is returned from the dialyzer 22 through extracorporeal tubing 24 and return needle or catheter 26. In some parts of the world, the extracorporeal blood flow may additionally receive a heparin drip to prevent clotting. The excess fluids and toxins are removed by clean dialysate liquid which is supplied to the dialyzer 22 via tube 28, and waste liquid is removed for disposal via tube 30. A typical hemodialysis treatment session takes about 3 to 5 hours in the United States.

The optical blood monitoring system 14 includes a display device 35 and a sensor device 34. The sensor device 34 may, for example, be a sensor clip assembly that is clipped to a blood chamber 32, wherein the blood chamber 32 is disposed in the extracorporeal blood circuit. A controller of the optical blood monitoring system 14 may be implemented in the display device 35 or in the sensor clip assembly 34, or both the display device 35 and the sensor clip assembly 34 may include a respective controller for carrying out respective operations associated with the optical blood monitoring system.

The blood chamber 32 may be disposed in line with the extracorporeal tubing 18 upstream of the dialyzer 22. Blood from the peristaltic pump 20 flows through the tubing 18 into the blood chamber 32. The sensor device 34 includes emitters that emit light at certain wavelengths and detectors for receiving the emitted light after it has passed through the blood chamber 32. For example, the emitters may include LED emitters which emit light at approximately 810 nm, which is isobestic for red blood cells, at approximately 1300 nm, which is isobestic for water, and at approximately 660 nm, which is sensitive for oxygenated hemoglobin, and the detectors may include a silicon photodetector for detecting light at the approximately 660 and 810 nm wavelengths, and an indium gallium arsenide photodetector for detecting light at the approximately 1300 nm wavelength. The blood chamber 32 includes lenses or viewing windows that allows the light to pass through the blood chamber 32 and the blood flowing therein.

A controller of the optical blood monitoring system 14 uses the light intensities measured by the detectors to determine Hct values for blood flowing through the blood chamber 32. The controller calculates hematocrit, oxygen saturation, and change in blood volume associated with blood passing through the blood chamber 32 to which the sensor device 34 is attached using a ratiometric model. The intensity of the received light at each of the various wavelengths is reduced by attenuation and scattering from the fixed intensity of the visible and infrared light emitted from each of the LED emitters. Beer's Law, for each wavelength of light, describes attenuation and scattering as follows:

iₙ=I₀₋ₙ*e^{-ε}^{ₚ}^{X}^{_{b}}^{d}^{ₚₜ}*e^{-ε}^{_{b}}^{X}^{_{b}}^{d}^{_{b}}*e^{-ε}^{ₚ}^{X}^{ₚ}^{d}^{ₚᵣ} Eq. (1)

where iₙ=received light intensity at wavelength n after attenuation and scattering; I₀₋ₙ=transmitted light intensity at wavelength n incident to the measured medium; e=the natural exponential term; ε=the extinction coefficient for the measured medium (p—blood chamber polycarbonate, b—blood); X=the molar concentration of the measured medium (p—blood chamber polycarbonate, b—blood); and d=the distance through the measured medium (pt—transmitting blood chamber polycarbonate, b—blood, pr—receiving blood chamber polycarbonate).

Since the properties of the polycarbonate blood chamber do not change, the first and third exponential terms in the above Eq. (1) are constants for each wavelength. Mathematically, these constant terms are multiplicative with the initial constant term I₀₋ₙ which represents the fixed intensity of the radiation transmitted from a respective LED emitter. For simplification purposes, Eq. (1) can be rewritten in the following form using bulk extinction coefficients and a modified initial constant I′₀₋ₙ as follows:

iₙ=I′₀₋ₙ*e^{-α}^{_{b}}^{d}^{_{b}} Eq. (2)

where iₙ=received light intensity at wavelength “n” after attenuation and scattering as though the detector were at the receive blood boundary; α=the bulk extinction coefficient (α_{b}=ε_{b}X_{b}) and I′₀₋ₙ=the equivalent transmitted light intensity at wavelength n as if applied to the transmit blood boundary accounting for losses through the blood chamber. Note that the term I′₀₋ₙ is the light intensity incident on the blood with the blood chamber losses included.

Using the approach defined in Eq. (2) above, the 810 nm wavelength which is isobestic for red blood cells and the 1300 nm wavelength which is isobestic for water can be used to determine the patient's hematocrit. The ratio of the normalized amplitudes of the measured intensity at these two wavelengths produces the ratio of the composite extinction values α for the red blood cells and the water constituents in the blood chamber, respectively. A mathematical function then defines the measured HCT value:

$$HCT = f [ \frac{ln ( \frac{{i}_{810}}{{I}_{0810}} )}{ln ( \frac{{i}_{1300}}{{I}_{01300}} )} ] \qquad Eq . ( 3 )$$

where i_{S10} is the light intensity of the photo receiver at 810 nm, i₁₃₀₀ is the infrared intensity of the photodetector at 1300 nm and I₀₋₈₁₀ and I₀₋₁₃₀₀ are constants representing the intensity incident on the blood accounting for losses through the blood chamber. The above equation holds true assuming that the flow of blood through the blood chamber 32 is in steady state, i.e. steady pressure and steady flow rate.

The preferred function f[ ] is a second order polynomial having the following form:

$$HCT = f [ \frac{ln ( \frac{{i}_{810}}{{I}_{0 - 810}} )}{ln ( \frac{{i}_{1300}}{{I}_{0 - 1300}} )} ] = {A [ \frac{ln ( \frac{{i}_{810}}{{I}_{0 - 810}} )}{ln ( \frac{{i}_{1300}}{{I}_{0 - 1300}} )} ]}^{2} + B [ \frac{ln ( \frac{{i}_{810}}{{I}_{0 - 810}} )}{ln ( \frac{{i}_{1300}}{{I}_{0 - 1300}} )} ] + C . \qquad Eq . ( 4 )$$

A second order polynomial is normally adequate as long as the infrared radiation incident at the first and second wavelengths is substantially isobestic.

After the Hct value has been determined by a controller at the sensor device 34 or at the display device 35, the display device may be used to output the determined Hct value. Further, the controller may further determine an Hgb concentration value based on the determined Hct value, with the Hgb concentration value also being output on the display device 35.

The hemodialysis system depicted in FIG. 2 may be one of a plurality of hemodialysis systems in a dialysis clinic. Patients may come into the dialysis clinic for treatments at regular intervals, for example, on a Monday-Wednesday-Friday schedule or a Tuesday-Thursday-Saturday schedule.

It will be appreciated that the hemodialysis system depicted in FIG. 2 is merely exemplary. The principles discussed herein are applicable to other types of hemodialysis systems having optical blood monitoring systems, as well as other types of dialysis systems and medical devices. The teachings of the present application with respect to determination of a calibrated Hgb concentration value are applicable to other medical systems in which blood monitoring operations are performed to measure Hct.

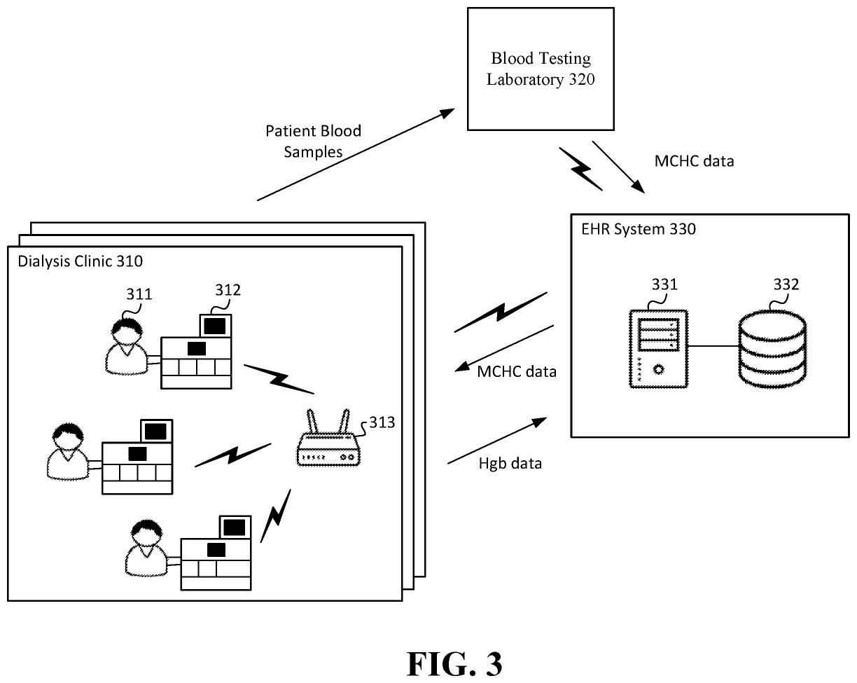

FIG. 3 is a block diagram of an exemplary network environment in which a hemodialysis system communicates with an electronic health records (EHR) system to obtain patient-specific MCHC data and determine a calibrated hemoglobin (Hgb) concentration value based on the patient-specific MCHC data. The network environment includes one or more dialysis clinics (including a respective dialysis clinic 310), a blood testing laboratory 320, and an EHR system 330.

The dialysis clinic 310 includes one or more hemodialysis systems used to provide hemodialysis treatment to one or more patients (including a respective patient 311 and a respective hemodialysis system 312). Each of the hemodialysis systems is in communication with a gateway device 313, for example, via a wired connection (e.g., an Ethernet RJ-45 connection or a fiber optic connection) or a wireless connection (e.g., via Bluetooth or WiFi). For example, a display device or a sensor device of an optical blood monitoring system of each of the hemodialysis systems may include a communications interface and corresponding communications equipment for communicating with the gateway device via the wired or wireless connection. The gateway device 313 is configured to communicate with an EHR system 330 over one or more networks (such as via a private computing network, via a public computing network such as the Internet, and/or via a mobile communications network). The EHR system 330 includes, for example, at least one application server 331 and at least one database 332 connected to the at least one application server 331. The EHR system 330 is configured, among other things, to store patient health information (e.g., pertaining to patient 311 and other patients being treated at the one or more dialysis clinics) in the at least one database 332 and to process and respond to requests for electronic health information via the at least one application server 331. The EHR system 330 receives patient health information from various sources, including the one or more dialysis clinics and from the blood testing laboratory 320, and the EHR system 330 may be configured to communicate with the various sources over one or more networks (such as via a private computing network, via a public computing network such as the Internet, and/or via a mobile communications network).

Patients undergoing dialysis treatments are regularly subjected to blood draws (e.g., at weekly or monthly intervals), whereby the blood samples obtained from the blood draws are sent to and analyzed by blood testing laboratories (such as blood testing laboratory 320). Patient health information determined from analyzing the patient blood samples is used to help make therapeutic decisions for the patients, including with respect to anemia management.

In accordance with exemplary embodiments of the present application, as shown in FIG. 3, patient blood samples obtained from a dialysis clinic 310 (or obtained at other medical facilities) are sent to the blood testing laboratory 320. The blood testing laboratory 320 analyzes the patient blood samples and sends MCHC data (among other health-related data) to EHR system 330. EHR system 330 maintains a record of the MCHC data (and other health-related data) for various patients, including historical MCHC data and other historical health-related data for each patient. Thus, prior to or during treatment of a patient 311 via a hemodialysis system 312 at dialysis clinic 310, the hemodialysis system 312 may send a request for patient-specific MCHC data of patient 311 to the EHR system 330 via gateway device 313. The EHR system 330 provides the patient-specific MCHC data for patient 311 to hemodialysis system 312, and hemodialysis system 312 is thus able to determine a calibrated Hgb concentration value for patient 311 using the patient-specific MCHC data and an Hct measurement. The dialysis clinic 310 may further provide Hgb data relating to patient 311 back to the EHR system 330 for storage and/or for further analysis or processing.

It will be appreciated that the network environment depicted in FIG. 3 is merely exemplary. The principles discussed herein are also applicable to other types of network configurations, entities, and equipment. The teachings of the present application with respect to determination of a calibrated Hgb concentration value are applicable to other network environments involving blood monitoring systems configured to determine Hct values.

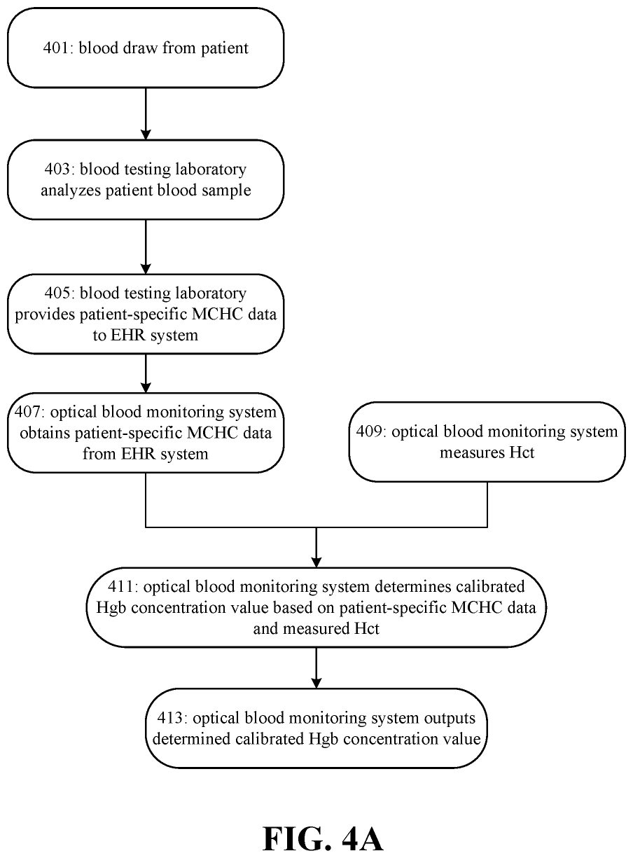

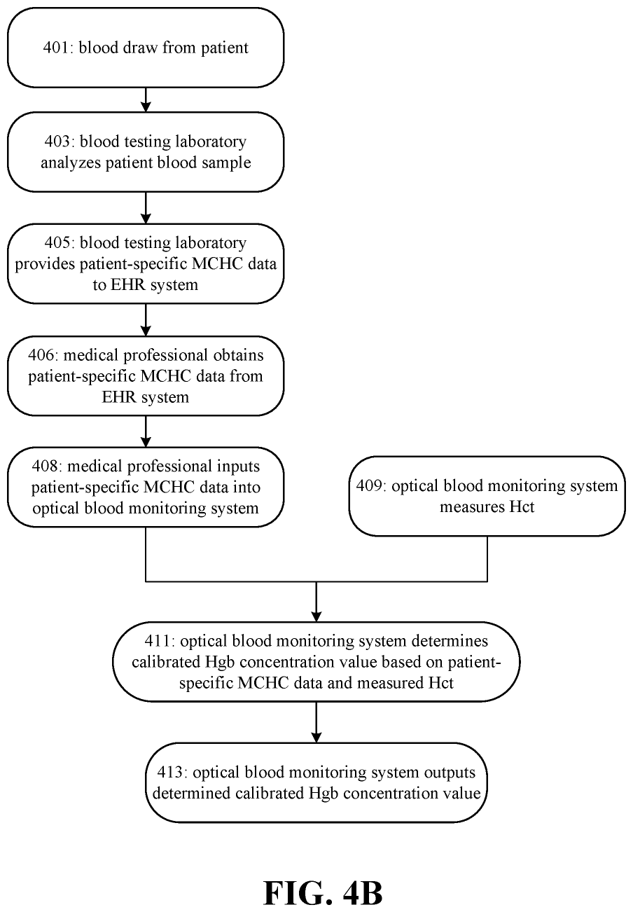

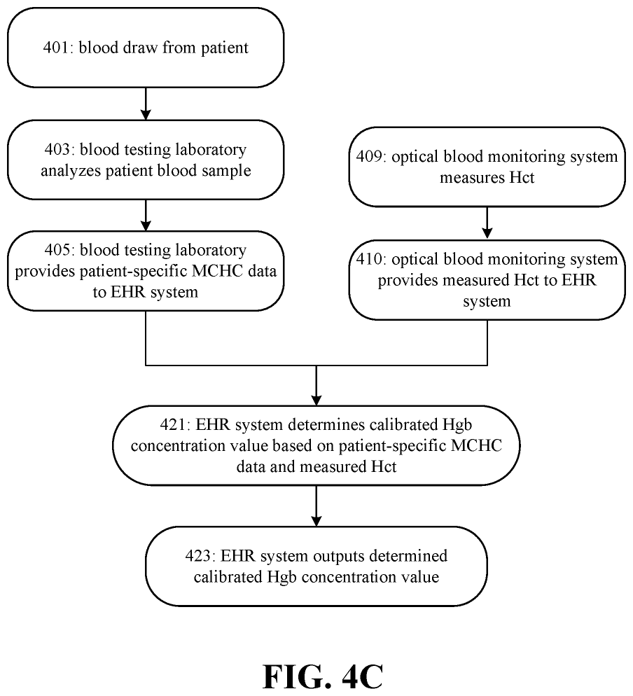

FIG. 4A is a flowchart of an exemplary process for determining a calibrated Hgb concentration value for a patient using an optical blood monitoring system.

At stage 401, a medical professional performs a blood draw on a patient to obtain a patient blood sample. This may be carried out, for example, at a dialysis clinic, and may be repeated on a periodic basis (e.g., weekly or monthly). The patient blood sample is then sent to a blood testing laboratory for analysis.

At stage 403, the blood testing laboratory analyzes the patient blood sample to determine various blood characteristics corresponding to the patient, including a patient-specific MCHC value for the patient.

At stage 405, the blood testing laboratory provides patient health information relating to the blood sample analysis to an EHR system (or to a healthcare provider which inputs the patient health information relating to the blood sample analysis into an EHR system). For example, a laboratory instrument may directly communicate directly reportable lab results to a laboratory information management system which is in communication with the EHR system or with the healthcare provider, or a lab technician may enter the patient health information into a computing device at the blood testing laboratory which uploads the patient health information to the EHR system through a communications network. The patient health information relating to the blood sample analysis uploaded to the EHR system includes the patient-specific MCHC value. The EHR system stores the patient-specific MCHC value, as well as other patient health information, including historical patient health information of the patient (such as past patient-specific MCHC values for the patient).

At stage 407, before or during an optical blood monitoring process for the patient (for example, an optical blood monitoring process performed by an optical blood monitoring system of a hemodialysis system in connection with hemodialysis treatment for the patient), the optical blood monitoring system obtains the patient-specific MCHC data from the EHR system. For example, the optical blood monitoring system may communicate with the EHR system over a communications network via a gateway device at a dialysis clinic, and send a request for the patient-specific MCHC data to the EHR system. The EHR system responds to the request by sending the patient-specific MCHC data to the optical blood monitoring system. The patient-specific MCHC data that is sent to the optical blood monitoring system may include, for example, a single most recently determined patient-specific MCHC value for the patient, a number of recently determined patient-specific MCHC values for the patient (e.g., the last two or three determined patient-specific MCHC values for the patient), multiple recently determined patient-specific MCHC values for the patient over a predetermined time period (e.g., all patient-specific MCHC values for the patient determined within the last three months), or an average patient-specific MCHC value for the patient (e.g., corresponding to an average of a number of recently determined patient-specific MCHC values for the patient or an average of all patient-specific MCHC values for the patient over a predetermined time period).

At stage 409, during the optical blood monitoring process for the patient (for example, in connection with hemodialysis treatment for the patient), the optical blood monitoring system measures an Hct value corresponding to the patient. The measured Hct value may be a real-time Hct value determined based on passing light of certain wavelengths through a blood chamber having extracorporeal blood of the patient flowing through it.

At stage 411, the optical blood monitoring system determines an Hgb concentration value specifically calibrated for the patient based the patient-specific MCHC data and the measured Hct value. For example, when the patient-specific MCHC data comprises a single most recently determined patient-specific MCHC value for the patient or an average patient-specific MCHC value for the patient, the determination of the calibrated Hgb concentration value may be based on a multiplication operation (or an equivalent division operation) using the patient-specific MCHC value for the patient and the measured Hct value. In another example, when the patient-specific MCHC data comprises multiple patient-specific MCHC values for the patient, the optical blood monitoring system may first determine an average patient-specific MCHC value for the patient and then use the determined average patient-specific MCHC value for the patient to determine the calibrated Hgb concentration value for the patient.

Further, as the optical blood monitoring system updates measured Hct values for patient in real-time, the optical blood monitoring system may correspondingly determine calibrated Hgb concentration values for the patient in real-time.

At stage 413, the optical blood monitoring system outputs the calibrated Hgb concentration value for the patient determined at stage 411. Outputting the calibrated Hgb concentration value may include displaying the calibrated Hgb concentration value on a display device of the optical blood monitoring system and/or transmitting the calibrated Hgb concentration value to another entity, such as the EHR system. The other entity and/or the optical blood monitoring system may further use the calibrated Hgb concentration value for a variety of treatment-related applications. For example, the Hgb data for the patient can be input into an algorithm-based anemia management model or an anemia management controller application based on a deterministic mathematical erythropoiesis model that ingests various data points for a patient (including the Hgb data for the patient) to output therapeutic recommendations for a patient. To provide another example, the Hgb data for the patient can be used by the optical blood monitoring system and/or another system to automatically detect medical conditions that may warrant further investigation based on identifying a trend in the Hgb data changing over time. A trend in Hgb over time that is disproportionate in relation to the concurrent anemia therapy may be a sign of conditions such as chronic bleeding, increased red blood cell turnover (eryptosis) or increased resistance to erythropoiesis-stimulating agents, all of which may warrant further medical investigation to identify the underlying cause.

Further, as the optical blood monitoring system updates and displays measured Hct values in real-time, the optical blood monitoring system may correspondingly update and display determined calibrated Hgb concentration values in real-time.

It will be appreciated that using an average patient-specific MCHC value based on multiple patient-specific MCHC values for a patient as discussed above in connection with stages 407 and 411 may be advantageous to mitigate the effects of a potential laboratory error related to analysis of one or more blood samples of the patient, as deviations caused by the error would be reduced based on the averaging. And because the kinetics for a patient's MCHC are relatively slow (e.g., significant changes take place over a time period on the order of months), including patient-specific MCHC data that is 2-3 months old in an average patient-specific MCHC value for the patient would not, on its own, cause the average patient-specific MCHC value for the patient to be significantly inaccurate or out-of-date.

FIG. 4B is a flowchart of another exemplary process for determining a calibrated Hgb concentration value for a patient using an optical blood monitoring system. FIG. 4B is similar to FIG. 4A except that FIG. 4B addresses a situation in which the optical blood monitoring system might not be able to obtain patient-specific MCHC data directly from the EHR system. In this situation, stages 406 and 408 of FIG. 4B may be performed instead of stage 407 of FIG. 4A. At stage 406, a medical professional (e.g., a clinician at a dialysis clinic) may use a computing device (e.g., a clinician device at the dialysis clinic) to access the EHR system and obtain patient-specific MCHC data from the EHR system. Then, at stage 408, the medical professional inputs the patient-specific MCHC data into the optical blood monitoring system, for example, via a graphical user interface of a display device of the optical blood monitoring system.

In yet another exemplary process, the medical professional may obtain the patient-specific MCHC data directly from a printed lab report (e.g., received by a dialysis clinic directly from a blood testing laboratory) and may input the patient-specific MCHC data from the printed lab report into the optical blood monitoring system.

FIG. 4C is a flowchart of another exemplary process for determining a calibrated Hgb concentration value for a patient using an optical blood monitoring system and an EHR system. Stages 401, 403, 405 and 409 of FIG. 4C are similar to stages 401, 403, 405 and 409 of FIGS. 4A and 4B. Further, in FIG. 4C, at stage 410, the optical blood monitoring system provides the measured Hct to the EHR system, and it is the EHR system which determines and outputs a calibrated Hgb concentration value at stages 421 and 423.

There may be situations in which patient-specific MCHC data is not available. For example, there may be a network outage such that an optical blood monitoring system cannot access patient-specific MCHC data from the EHR system. Thus, stage 411 may further include determining whether or not patient-specific MCHC data is available. In the case where patient-specific MCHC data is not available, a default MCHC value may be used. In one exemplary embodiment, the default MCHC value may be a single MCHC value used for all patients, corresponding to an average MCHC value determined across a large patient population. In another exemplary embodiment, the default MCHC value may be chosen from one of several demographic-specific MCHC values based on a characteristic of a patient. For example, there may be one default MCHC value for males and another default MCHC value for females. To provide another example, respective clinics may use clinic-specific default MCHC values for each respective clinic (representing all patients of the clinic) which are periodically updated. To provide yet another example, different default MCHC values may correspond to different altitude ranges. In yet another exemplary embodiment, during a network outage, the optical blood monitoring system may prompt a user to input an MCHC value (e.g., an MCHC value for a specific patient which is obtained from the patient's chart).

Exemplary embodiments of the present application provide for improved accuracy with respect to determination of Hgb concentration values for patients using blood monitoring systems based on measured Hct and patient-specific MCHC values. This in turn improves the reliability and usability of Hgb data determined by the blood monitoring system for various purposes, such as for making anemia management decisions and for detecting chronic bleeding. Further, it may be possible to reduce the frequency at which a patient's blood is drawn as a result of reliable Hgb data being available through the blood monitoring system.

It will be appreciated that although the exemplary embodiments discussed above include an optical blood monitoring system, the principles discussed herein are also applicable to blood monitoring systems which utilize other types of technology to measure Hct (for example, via acoustic monitoring, via magnetic resonance imaging using a native R1 (inverse T1) value of blood, or via electrical admittance plethysmography).

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

All references, including publications, patent applications, and patents, cited herein are hereby incorporated by reference to the same extent as if each reference were individually and specifically indicated to be incorporated by reference and were set forth in its entirety herein.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present application covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A hemodialysis system, comprising:
a hemodialysis machine configured to provide hemodialysis treatment to a patient, wherein the hemodialysis treatment includes circulating extracorporeal blood of the patient through an extracorporeal blood circuit; and
a blood monitoring system, comprising:
a sensor device configured to measure a hematocrit value corresponding to the extracorporeal blood of the patient in the extracorporeal blood circuit; and
at least one controller;
wherein the blood monitoring system is configured to communicate with an electronic health records (EHR) system over a communications network to obtain patient-specific mean corpuscular hemoglobin concentration (MCHC) data for the patient; and
wherein the at least one controller is configured to determine a hemoglobin concentration value corresponding to the extracorporeal blood for the patient in the extracorporeal blood circuit using the measured hematocrit value and the patient-specific MCHC data for the patient.

2. The hemodialysis system according to claim 1, wherein the at least one controller of the blood monitoring system comprises a controller disposed within the sensor device.

3. The hemodialysis system according to claim 1, wherein the blood monitoring system further comprises a display device configured for displaying the measured hematocrit value and the determined hemoglobin concentration value.

4. The hemodialysis system according to claim 3, wherein the at least one controller of the blood monitoring system comprises a controller disposed within the sensor device.

5. The hemodialysis system according to claim 1, wherein the patient-specific MCHC data for the patient comprises multiple MCHC values;
wherein the at least one controller is further configured to determine an average MCHC value from the multiple MCHC values; and
wherein determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

6. The hemodialysis system according to claim 1, wherein the patient-specific MCHC data for the patient comprises a most recent MCHC value determined for the patient based on laboratory analysis of a blood sample of the patient.

7. The hemodialysis system according to claim 1, wherein the patient-specific MCHC data for the patient comprises an average MCHC value determined for the patient based on laboratory analyses of multiple blood samples of the patient.

8. The hemodialysis system according to claim 1, wherein determining the hemoglobin concentration value corresponding to the extracorporeal blood of the patient in the extracorporeal blood circuit comprises performing a multiplication or division operation using the measured hematocrit value and an MCHC value corresponding to the patient-specific MCHC data for the patient.

9. The hemodialysis system according to claim 1, wherein the blood monitoring system is further configured to communicate with the EHR system to provide the determined hemoglobin concentration value to the EHR system.

10. The hemodialysis system according to claim 1, wherein the blood monitoring system further comprises a user interface for receiving input of a patient-specific MCHC value for the patient; and
wherein the at least one controller is further configured to determine a hemoglobin concentration value corresponding to the extracorporeal blood of the patient in the extracorporeal blood circuit using the measured hematocrit value and the input patient-specific MCHC value for the patient.

11. The hemodialysis system according to claim 1, wherein the blood monitoring system is configured to update the measured hematocrit value in real-time; and
wherein the at least one controller is configured to correspondingly update the determined hemoglobin concentration value in real-time.

12. The hemodialysis system according to claim 1, wherein the blood monitoring system is an optical blood monitoring system.

13. A method for monitoring blood using a blood monitoring system, comprising:
measuring, by a sensor device of the blood monitoring system, a hematocrit value of extracorporeal blood of a patient in an extracorporeal circuit to which the sensor device is attached;
obtaining, by at least one controller of the blood monitoring system, patient-specific mean corpuscular hemoglobin concentration (MCHC) data for the patient;
determining, by the at least one controller of the blood monitoring system, a hemoglobin concentration value corresponding to the extracorporeal blood of the patient in the extracorporeal blood circuit using the measured hematocrit value and the patient-specific MCHC data for the patient; and
outputting, by the at least one controller of the blood monitoring system, the determined hemoglobin concentration value.

14. The method according to claim 13, wherein obtaining the patient-specific MCHC data for the patient further comprises:
obtaining, by at least one controller of the blood monitoring system, the patient-specific MCHC data from an electronic health records (EHR) system via a communications interface of the blood monitoring system.

15. The method according to claim 13, wherein the patient-specific MCHC data comprises multiple MCHC values;
wherein the method further comprises: determining, by the at least one controller of the blood monitoring system, an average MCHC value from the multiple MCHC values; and
wherein determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

16. The method according to claim 13, wherein the patient-specific MCHC data for the patient comprises a most recent MCHC value determined for the patient based on laboratory analysis of a blood sample of the patient.

17. The method according to claim 13, wherein the patient-specific MCHC data for the patient comprises an average MCHC value determined for the patient based on laboratory analyses of multiple blood samples of the patient.

18. The method according to claim 13, wherein the blood monitoring system is an optical blood monitoring system.

19. A non-transitory computer-readable medium having processor-executable instructions stored thereon for monitoring blood using a blood monitoring system, the processor-executable instructions, when executed, facilitating:
measuring a hematocrit value of extracorporeal blood of a patient in an extracorporeal circuit to which the sensor device is attached;
obtaining patient-specific mean corpuscular hemoglobin concentration (MCHC) data for the patient;
determining a hemoglobin concentration value corresponding to the extracorporeal blood of the patient in the extracorporeal blood circuit using the measured hematocrit value and the patient-specific MCHC data for the patient; and
outputting the determined hemoglobin concentration value.

20. The non-transitory computer-readable medium according to claim 19, wherein obtaining the patient-specific MCHC data for the patient further comprises:
obtaining the patient-specific MCHC data from an electronic health records (EHR) system via a communications interface of the blood monitoring system.

21. The non-transitory computer-readable medium according to claim 19, wherein the patient-specific MCHC data comprises multiple MCHC values;
wherein the processor-executable instructions, when executed, further facilitate: determining an average MCHC value from the multiple MCHC values; and
wherein determining the hemoglobin concentration value corresponding to the extracorporeal blood of the patient in the extracorporeal blood circuit uses the measured hematocrit value and the average MCHC value determined from the multiple MCHC values of the patient-specific MCHC data for the patient.

22. The non-transitory computer-readable medium according to claim 19, wherein the patient-specific MCHC data for the patient comprises a most recent MCHC value determined for the patient based on laboratory analysis of a blood sample of the patient.

23. The non-transitory computer-readable medium according to claim 19, wherein the patient-specific MCHC data for the patient comprises an average MCHC value determined for the patient based on laboratory analyses of multiple blood samples of the patient.

24. The non-transitory computer-readable medium according to claim 19, wherein the blood monitoring system is an optical blood monitoring system.
